(19) United States
(12) Patent Application Publication
(10) Pub. No.: US 2007/0254947 A1
(43) Pub. Date: Nov. 1, 2007

(54) Composition for external use

(75) Inventors: Kumiko Takiguchi, Osaka-shi (JP); Norikazu Sato, Osaka-shi (JP)

Correspondence Address: SUGHRUE MION, PLLC, 2100 PENNSYLVANIA AVENUE, N.W., SUITE 800, WASHINGTON, DC, 20037, US

(73) Assignee: ROHTO PHARMACEUTICAL CO., LTD., Osaka-shi (JP)

(21) Appl. No.: 11/790,773

(22) Filed: Apr. 27, 2007

(30) Foreign Application Priority Data
Apr. 28, 2006 (JP) 2006-125081

(51) Int. Cl. A61K 31/355; A61K 31/05

(52) U.S. Cl. 514/458; 514/734

(57) ABSTRACT

An object of the present invention is to provide a hydroquinone-containing composition for external use in which cytotoxicity and skin irritating properties are reduced.

The composition for external use of the present invention is prepared by adding a tocopherol together with hydroquinone. Among tocopherols, γ- and δ-tocopherols are particularly effective for achieving the above object. The composition can be used as a cosmetic composition, skin cleansing composition, or pharmaceutical composition.

### BACKGROUND OF THE INVENTION

(1) Field of the Invention

The present invention relates to a composition for external use in which the cytotoxicity of hydroquinone is reduced.

(2) Description of the Related Art

Hydroquinone is known to have strong melanogenesis inhibitory effects and to be a useful component of skin-whitening compositions for external use. Hydroquinone, however, has the drawbacks of cytotoxicity and skin irritation, and it is essential to overcome such drawbacks when preparing hydroquinone-containing compositions for external use.

It has been reported that substances having catalase-like actions, such as ascorbic acid and the like, have an effect of alleviating the above drawbacks of hydroquinone; however, such substances have problems of low persistence of the above effect, low stability in compositions, etc., and are not yet satisfactory.

Tocopherols are known to eliminate or inactivate a radical of peroxidized lipid and active oxygen to thereby exhibit antioxidant effects. Tocopherols are methylated derivatives of tocol, and four different structures of tocopherols with methyl groups at different positions, i.e., α-tocopherol (5,7,8-trimethyltocol), β-tocopherol (5,8-dimethyltocol), γ-tocopherol (7,8-dimethyltocol), and δ-tocopherol (8-methyltocol) are known. Reportedly, the ratio of biological activities of α-, β-, γ-, and δ-tocopherols is approximately 100:24:4:0.1, while the antioxidant activity against oils and fats is highest in δ-tocopherol and lowest in α-tocopherol (J. Act. Oxyg. Free Rad. 3:531-54). However, there has been no report on catalase-like actions of tocopherols, and the prior art does not even suggest how tocopherols act on hydroquinone.

### BRIEF SUMMARY OF THE INVENTION

An object of the present invention is to provide a hydroquinone-containing composition for external use in which the cytotoxicity and skin irritating properties of hydroquinone are reduced. Another object of the present invention is to provide a process for producing the composition for external use.

The present inventors conducted extensive research to solve the above problems, and found that tocopherols reduce the cytotoxicity of hydroquinone. The present invention was accomplished by further research based on the above finding.

The present invention provides the following compositions for external use.

Item 1. A composition for external use comprising hydroquinone and at least one tocopherol.

Item 2. A composition according to item 1, wherein said least one tocopherol is selected from the group consisting of α-tocopherol, γ-tocopherol, and δ-tocopherol.

Item 3. A composition according to item 1, wherein said at least one tocopherol is selected from the group consisting of γ-tocopherol and δ-tocopherol.

Item 4. A composition according to item 1, comprising 0.0005 to 10 wt. % of hydroquinone and 0.01 to 10 wt. % of tocopherol, based on the total amount of the composition.

Item 5. A composition according to item 1, comprising 0.01 to 12 parts by weight of tocopherol per part by weight of hydroquinone.

Item 6. A composition according to item 1, which is a cosmetic composition.

Item 7. A composition according to item 1, which is a skin cleansing composition.

Item 8. The composition according to item 1, which is a pharmaceutical composition.

The present invention further provides the following processes for producing a composition for external use.

Item 9. A process for producing a composition for external use, the process comprising the step of adding hydroquinone and at least one tocopherol to a pharmaceutically or cosmetically acceptable carrier.

Item 10. A process according to item 9, wherein said at least one tocopherol is selected from the group consisting of α-tocopherol, γ-tocopherol, and δ-tocopherol.

Item 11. A process according to item 9, wherein said least one tocopherol is selected from the group consisting of γ-tocopherol and δ-tocopherol.

Item 12. A process according to item 9, wherein the hydroquinone and tocopherol are added in proportions of 0.0005 to 10 wt. % and 0.01 to 10 wt. %, respectively, based on the total amount of the composition.

Item 13. A process according to item 9, wherein the hydroquinone and tocopherol are added in a ratio of 0.01 to 12 parts by weight of tocopherol per part by weight of hydroquinone.

Item 14. A process according to item 9, wherein the composition for external use is a cosmetic composition.

Item 15. The process according to item 9, wherein the composition for external use is a skin cleansing composition.

Item 16. The process according to item 12, wherein the composition for external use is a pharmaceutical composition.

The present invention also provides the following methods for reducing cytotoxicity.

Item 17. A method for reducing cytotoxicity of a hydroquinone-containing composition for external use, the method comprising addition of at least one tocopherol to the composition.

Item 18. A method according to item 17, wherein said at least one tocopherol is selected from the group consisting of α-tocopherol, γ-tocopherol, and δ-tocopherol.

Item 19. A method according to item 17, wherein said least one tocopherol is selected from the group consisting of γ-tocopherol and δ-tocopherol.

Item 20. A method according to item 17, wherein the composition for external use contains 0.0005 to 10 wt. % of hydroquinone based on the total amount of the composition.

Item 21. A method according to item 17, wherein the tocopherol is added in a proportion of 0.01 to 10 wt. % based on the total amount of the composition.

Item 22. A method according to item 17, wherein the tocopherol is added in a ratio of 0.01 to 12 parts by weight per part by weight of hydroquinone contained in the composition for external use.

Item 23. A method according to item 17, wherein the composition for external use is a cosmetic composition.

Item 24. A method according to item 17, wherein the composition for external use is a skin cleansing composition.

Item 25. A method according to item 17, wherein the composition for external use is a pharmaceutical composition.

Further, the present invention also provides the following methods for reducing cytotoxicity of hydroquinone on the skin.

Item 26. A method for reducing cytotoxicity of hydroquinone on the skin, the method comprising applying, to the skin, a composition (A) containing hydroquinone and a composition (B) containing at least one tocopherol.

Item 27. A method according to item 26, wherein the composition (A) is applied to the skin after applying the composition (B).

Item 28. A method according to item 26, wherein said at least one tocopherol is selected from the group consisting of α-tocopherol, γ-tocopherol, and δ-tocopherol.

Item 29. A method according to item 26, wherein said least one tocopherol is selected from the group consisting of γ-tocopherol and δ-tocopherol.

Item 30. A method according to item 26, wherein the composition (A) contains 0.0005 to 10 wt. % of hydroquinone based on the total amount of the composition.

Item 31. A method according to item 26, wherein the composition (B) contains 0.01 to 10 wt. % of the tocopherol based on the total amount of the composition.

Item 32. A method according to item 16, wherein the compositions (A) and (B) are applied in such a manner that the tocopherol become in a ratio of 0.01 to 12 parts by weight per part by weight of hydroquinone.

Item 33. A method according to item 26, wherein the compositions (A) and (B) are cosmetic compositions.

Item 34. A method according to item 26, wherein wherein the compositions (A) and (B) are skin cleansing compositions.

Item 25. A method according to item 17, wherein wherein the compositions (A) and (B) are pharmaceutical compositions.

The present invention was accomplished based on the findings that tocopherols have an effect of reducing the cytotoxicity of hydroquinone, and that such an effect of γ-tocopherol and δ-tocopherol is higher than that of α-tocopherol, which is said to have the highest physiological activity among known tocopherols.

Further, the excellent effects of hydroquinone, such as melanogenesis inhibition and the like, are expected to be obtained by adding hydroquinone and tocopherol(s). Therefore, according to the present invention, it is possible to provide a safe and useful hydroquinone-containing composition for external use.

### DETAILED DESCRIPTION OF THE INVENTION

### 1. Composition for External Use

The composition for external use of the present invention comprises hydroquinone and at least one tocopherol.

Hydroquinone is a known compound used in the field of pharmaceuticals, quasi drugs, or cosmetics.

In the composition of the present invention, the proportion of hydroquinone can be suitably selected considering the feel and effects on the skin, and is, for example, 0.0005 to 10 wt. %, preferably 0.0008 to 8 wt. %, and more preferably 0.001 to 6 wt. %, of the total amount of the composition.

The tocopherol for use in the composition of the present invention can be obtained by a standard method, and may be a commercially available tocopherol. Four structures of tocopherols, i.e., α-, β-, γ-, and δ-tocopherols, are known, and any of these tocopherols can be used in the present invention. α-, γ-, and δ-tocopherols are preferable, with γ- and δ-tocopherols being especially preferable. These tocopherols can be used singly or in combination.

In the composition of the present invention, the proportion of tocopherol(s) is not limited as long as the hydroquinone-cytotoxicity-reducing effect is achieved, and can be suitably selected considering the feel and effects on the skin. The proportion may be, for example, 0.01 to 10 wt. %, preferably 0.1 to 8 wt. %, and more preferably 1 to 6 wt. %., of the total amount of the composition.

In the composition of the present invention, the proportion of tocopherol(s) to hydroquinone is not limited as long as it is selected according to the above-mentioned proportions of hydroquinone and tocopherol(s). From the viewpoint of more effective reduction of the cytotoxicity of hydroquinone, it is preferable to use tocopherol(s) in a ratio of, for example, 0.01 to 12 parts by weight, preferably 0.05 to 6 parts by weight, and more preferably 0.1 to 4 parts by weight, per part by weight of hydroquinone.

To stabilize hydroquinone, the composition of the present invention may contain at least one stabilizing component selected from the group consisting of glycols, glycol ethers, glycerol, and diglycerol.

Glycols that can be used as stabilizing components include diols that are liquid at 25° C. and are used in the field of pharmaceuticals, quasi drugs, or cosmetics. Specific examples of diols include diols represented by the formula CₙH₂ₙ(OH)₂, and condensates of one or more of these diols, etc. More specific examples include ethylene glycol, propylene glycol, trimethylene glycol, 1,2-butylene glycol, 1,3-butylene glycol, 2,3-butylene glycol, isoprene glycol, 1,2-pentylene glycol, 1,2-hexylene glycol, octylene glycol, etc.; and condensates such as diethylene glycol, triethylene glycol, tetraethylene glycol, dipropylene glycol, tripropylene glycol, etc. Among these, propylene glycol, 1,3-butylene glycol, and dipropylene glycol are preferable.

Glycol ethers that can be used as stabilizing components are not limited as long as they are compounds obtained by etherification of one or both glycol hydroxy groups and are used in the field of pharmaceuticals, quasi drugs, or cosmetics. Glycol residues that form glycol ethers are diols that are liquid at 25° C. Examples thereof include residues of diols represented by the formula CₙH₂ₙ(OH)₂, and residues of condensates of one or more of these diols.

Specific examples of glycol ethers include ethylene glycol monomethyl ether, ethylene glycol monoethyl ether, ethylene glycol monopropyl ether, diethylene glycol monomethyl ether, diethylene glycol monoethyl ether, diethylene glycol monopropyl ether, diethylene glycol monobutyl ether, propylene glycol monoethyl ether, propylene glycol monopropyl ether, dipropylene glycol monoethyl ether, and dipropylene glycol monopropyl ether, among which diethylene glycol monoethyl ether and diethylene glycol monobutyl ether are especially preferable.

Glycerol and diglycerol that can be used as the stabilizing components are known compounds that are used in compositions for external use.

The above stabilizing components may be used singly or in combination.

Specifically, preferable examples of stabilizing components are (i) at least one member selected from propylene glycol, 1,3-butylene glycol, dipropylene glycol, diethylene glycol monoethyl ether, glycerol, and diglycerol. Another preferable example of stabilizing component is (ii) at least one glycol, and specifically, (ii) at least one member selected from propylene glycol, 1,3-butylene glycol, and dipropylene glycol.

The proportion of such stabilizing component(s) in the composition of the present invention is not limited as long as the effects of the present invention are not impaired. The total content of stabilizing component(s) may be, for example, 5 to 90 wt. %, preferably 10 to 80 wt. %, and more preferably 20 to 70 wt. %, of the total amount of the composition.

To enhance or supplement the effects of the present invention, the composition of the present invention may contain at least one member selected from anti-aging components, moisturizing components, skin-whitening components, anti-inflammatory components, antimicrobial components, cell-activating components, astringent components, and antioxidant components. These components are not limited and can be arbitrarily used as long as they are components that are used or will be used in the future as components of external preparations for skin in the field of pharmaceuticals, quasi drugs, or cosmetics. In the present invention, particularly preferable combinations of such components include combinations of tocopherols and moisturizing components; combinations of tocopherols and skin-whitening components; combinations of tocopherols, moisturizing components, and antioxidant components; combinations of tocopherols and anti-aging components; combinations of tocopherols, anti-aging components, and antioxidant components; and combinations of tocopherols, cell-activating components, and anti-aging components.

Examples of anti-aging components include retinoids (retinol, retinoic acid, retinal, etc.), pangamic acid, kinetin, ursolic acid, turmeric extracts, sphingosine derivatives, silicon, silicic acid, N-methyl-L-serine, mevalonolactone, etc., among which retinoids (retinol, retinoic acid, retinal, etc.) and kinetin are preferable. Such anti-aging components can be used singly or in combination.

When using anti-aging component(s), the proportion thereof is not limited as long as the effects of the present invention are exhibited, and can be suitably selected considering the feel and effects on the skin. The proportion of anti-aging component(s) is usually 0.0003 to 10 wt. %, and preferably 0.01 to 5 wt. %, of the total amount of the composition.

Examples of moisturizing components include alanine, serine, leucine, isoleucine, threonine, glycine, proline, hydroxyproline, glucosamine, theanine, and like amino acids, and derivatives thereof; gelatin and like peptides; glycerol, diglycerol, 1,3-butylene glycol, propylene glycol, polyethylene glycol, and like polyhydric alcohols; ethylene glycol monomethyl ether, ethylene glycol monoethyl ether, ethylene glycol monopropyl ether, diethylene glycol monomethyl ether, diethylene glycol monoethyl ether, diethylene glycol monopropyl ether, diethylene glycol monobutyl ether, propylene glycol monoethyl ether, propylene glycol monopropyl ether, dipropylene glycol monoethyl ether, dipropylene glycol monopropyl ether, and like glycol ethers; sorbitol and like sugar alcohols; lecithins (including soybean phospholipids), hydrogenated lecithins, and like phospholipids; heparin, chondroitin, and like mucopolysaccharides; lactic acid, sodium pyrrolidone carboxylate, urea, and like natural moisturizing factors (NMFs); polyglutamic acid; etc. Among such moisturizing components, preferable examples are alanine, serine, glycine, proline, hydroxyproline, glucosamine, theanine, glycerol, diglycerol, 1,3-butylene glycol, hydrogenated lecithins, heparin, chondroitin, lactic acid, sodium pyrrolidone carboxylate, and polyglutamic acid. Such moisturizing components can be used singly or in combination.

When using moisturizing component(s), the proportion thereof is not limited as long as the effects of the present invention are exhibited, and can be suitably selected considering the feel and effects on the skin. The proportion of moisturizing component(s) is usually 0.1 to 30 wt. %, preferably 0.5 to 10 wt. %, and more preferably 0.5 to 5 wt. %, of the total amount of the composition.

Examples of skin-whitening components include ellagic acid; phytic acid; rucinol; chamomile ET; vitamin A or derivatives thereof, vitamin C or derivatives thereof (such as magnesium ascorbyl phosphate), pantothenic acid or derivatives thereof, and like vitamins; etc. Among these, preferable examples are pantothenic acid or derivatives thereof, ellagic acid, phytic acid, vitamin A or derivatives thereof, and vitamin C or derivatives thereof; with vitamin C or derivatives thereof (such as magnesium ascorbyl phosphate) being particularly preferable. Such skin-whitening components can be used singly or in combination.

Plant components having skin-whitening effects can be used as skin-whitening components. Examples of such plant components include those derived from, for example, iris, almond, aloe, ginkgo, oolong tea, rose fruit, scutellaria root, Coptis Rhizome, St. John's wort (Hypericum erectum Thunb), dead nettle, seaweed, pueraria root, gardenia, Sophorae Radix, chlorella, gallnut, wheat, rice, rice germ, orizanol, rice bran, Asiasari Radix, zanthoxylum fruit, perilla, peony root, Cnidium Rhizome, mulberry bark, soybeans, fermented soybeans, tea, Japanese angelica, Calendula officinalis, garlic, hamamelis, safflower, moutan bark, Angelica acutiloba Kitagawa, amethyst, gambir, Japanese andromeda, Japanese andromeda, bracken, Podocarpus macrophyllus, Celtis sinensis, persimmon (Diospyros kaki), catalpa, black soybeans, gentian, figwort (Scrophularia ningpoensis), sarsaparilla, French beans, cimicifuga rhizome, Paris polyphylla Smith, sage, Peucedani Radix, Japanese radish, azalea, Lespedeza homoloba, seeds of Cuscuta chinensis Lam., Picrasma quassioides, parsley, hollies, hops, Lespedeza cyrtobotrya, cloves, licorice, etc. Preferable examples are components derived from plants such as iris, aloe, ginkgo, oolong tea, rose fruit, scutellaria root, Coptis Rhizome, St. John's wort (Hypericum erectum Thunb), dead nettle, seaweed, pueraria root, gardenia, Sophorae Radix, gallnut, wheat, rice, rice bran, Asiasari Radix, zanthoxylum fruit, perilla, peony, Cnidium Rhizome, mulberry bark, tea, Japanese angelica, Calendula officinalis, hamamelis, safflower, Moutan bark, coix seeds, Angelica acutiloba Kitagawa, gambir, Celtis sinensis, persimmon (Diospyros kaki), catalpa, black soybeans, gentian, sarsaparilla, French beans, Paris polyphylla Smith, sage, Peucedani Radix, Japanese radish, azalea, Lespedeza homoloba, seeds of Cuscuta chinensis Lam., Picrasma quassioides, parsley, hollies, hops, cloves, licorice and Japanese angelica. More preferable examples are components derived from plants such as iris, aloe, ginkgo, rose fruit, scutellaria root, Coptis Rhizome, St. John's wort (Hypericum erectum Thunb), gardenia, Sophorae Radix, rice, rice bran, Asiasari Radix, peony, Cnidium Rhizome, mulberry bark, tea, Japanese angelica, Calendula officinalis, hamamelis, safflower, moutan bark, amethyst, Gambir, Celtis sinensis, persimmon (Diospyros kaki), sage, Japanese radish, azalea, parsley, hops, licorice and coix seeds, etc. Such plant components can be used singly or in combination.

When using plant component(s) in the composition of the present invention, the form thereof is not limited, and may be a plant essence (plant extract), essential oil, etc. In the above list of plant components, either or both of general names and scientific names of the plants are shown.

When using skin-whitening component(s) other than plant components as mentioned above, the proportion thereof is preferably 0.0003 to 10 wt. %, and more preferably 0.01 to 5 wt. %, of the total amount of the composition.

When using plant component(s) as skin-whitening components, the proportion thereof, calculated as extract such as essence or essential oil, is usually 0.00001 to 20 wt. %, preferably 0.0001 to 15 wt. %, and more preferably 0.001 to 10 wt. %, of the total amount of the composition.

Specific examples of anti-inflammatory components include allantoin, calamine, glycyrrhizinic acid or derivatives thereof, glycyrrhetinic acid or derivatives thereof, zinc oxide, guaiazulene, tocopherol acetate, pyridoxine hydrochloride, menthol, camphor, turpentine oil, indomethacin, salicylic acid or derivatives thereof, etc. Among such components, preferable examples are allantoin; glycyrrhizinic acid or derivatives thereof, glycyrrhetinic acid or derivatives thereof, guaiazulene, and menthol. Such anti-inflammatory components can be used singly or in combination.

When using anti-inflammatory component(s), the proportion thereof is preferably 0.0003 to 10 wt. %, and more preferably 0.01 to 5 wt. %, of the total amount of the composition.

Specific examples of antimicrobial components include chlorhexidine, salicylic acid, benzalkonium chloride, acrinol, ethanol, benzethonium chloride, cresol, gluconic acid or derivatives thereof, povidone-iodine, potassium iodide, iodine, isopropyl methylphenol, triclocarban, triclosan, Photosensitizing Dye No. 101 (2,2′-[3-[2-(3-heptyl-4-methyl-4-thiazolin-2-ylidene)ethylidene]propenylene]bis(3-heptyl-4-methylthiazolinium iodide), Photosensitizing Dye No. 201 (3-heptyl-2-[3-heptyl-4-methyl-(3H)-thiazol-2-ylidene]methyl)-4-methylthiazolium iodide), parabens (methylparaben, propylparaben, butylparaben, etc.), phenoxyethanol, 1,2-pentanediol, alkyldiaminoglycine hydrochloride, etc. Preferable examples include benzalkonium chloride, benzethonium chloride, gluconic acid or derivatives thereof, isopropyl methylphenol, triclocarban, triclosan, Photosensitizing Dye No. 101, Photosensitizing Dye No. 201, parabens (methylparaben, propylparaben, butylparaben), phenoxyethanol, 1,2-pentanediol, alkyl diaminoglycine hydrochloride, etc. More preferable examples include benzalkonium chloride, gluconic acid or derivatives thereof, benzethonium chloride, and isopropyl methylphenol. Such antimicrobial components can be used singly or in combination.

When using antimicrobial component(s), the proportion thereof is preferably 0.0003 to 10 wt. %, and more preferably 0.01 to 5 wt. %, of the total amount of the composition.

Specific examples of cell-activating components include γ-aminobutyric acid, ε-aminocaproic acid, and like amino acids; retinol, thiamine, riboflavin, pyridoxine hydrochloride, pantothenic acid, and like vitamins; glycolic acid, lactic acid, and like α-hydroxy acids; tannins, flavonoids, saponins, allantoin, Photosensitizing Dye No. 301 (2-[2-[(5-bromo-2-pyridinyl)amino]vinyl]-1-ethyl-6-methylpyridinium iodide), etc. Preferable examples include γ-aminobutyric acid, ε-aminocaproic acid, and like amino acids; and retinol, thiamine, riboflavin, pyridoxine hydrochloride, pantothenic acid, and like vitamins. Such cell-activating components can be used singly or in combination.

When using cell-activating component(s), the proportion thereof is preferably 0.0003 to 10 wt. %, and more preferably 0.01 to 5 wt. %, of the total amount of the composition.

Specific examples of astringent components include alum, chlorohydroxyaluminum, aluminium chloride, aluminum salt of allantoin, zinc sulfate, aluminum potassium sulfate, and like metal salts; and tannic acid, citric acid, lactic acid, succinic acid, and like organic acids. Preferable examples include alum, chlorohydroxyaluminum, aluminium chloride, aluminum salt of allantoin, aluminum potassium sulfate, and tannic acid. Such astringent components can be used singly or in combination.

When using astringent component(s), the proportion thereof is usually 0.0003 to 10 wt. %, and preferably 0.01 to 5 wt. %, of the total amount of the composition.

Specific examples of antioxidant components include ascorbic acid or derivatives thereof (magnesium ascorbate phosphate, ascorbyl tetraisopalmitate, etc.), butylhydroxyanisole, dibutylhydroxytoluene, sodium hydrogen sulfite, .erythorbic acid and salts. thereof, flavonoids, catalase, superoxide dismutase, taurine, thiotaurine, hypotaurine, thioredoxin, flavonoid, catechin, astaxanthin, etc. Preferable examples include ascorbic acid or derivatives thereof, thiotaurine, hypotaurine, thioredoxin, flavonoids, and astaxanthin.. Such antioxidant components can be used singly or in combination.

When using antioxidant component(s), the proportion thereof is usually 0.00001 to 10 wt. %, preferably 0.0001 to 5 wt. %, and more preferably 0.001 to 2 wt. %, of the total amount of the composition.

The composition of the present invention may further contain surfactants, gelling agents, oils, fats, chelating agents, saccharides, UV protection agents, and/or the like, in addition to the above components.

Usable surfactants include, for example, polyoxyethylene (hereinafter sometimes referred to as POE)-octyldodecyl alcohol, POE-2-decyltetradecyl alcohol, and like POE-branched alkyl ethers; POE-oleyl ether, POE-cetyl ether, and like POE-alkyl ethers; sorbitan monooleate, sorbitan monoisostearate, sorbitan monolaurate, and like sorbitan esters; POE-sorbitan monooleate, POE-sorbitan monoisostearate, POE-sorbitan monolaurate, POE-sorbitan tetraoleate, and like POE-sorbitan esters; glycerol monooleate, glycerol monostearate, glycerol monomyristate, and like glycerol fatty-acid esters; POE-glycerol monooleate, POE-glycerol monostearate, POE-glycerol monomyristate,, and like POE-glycerol fatty acid esters; POE-dihydrocholesterol ester, POE-hydrogenated castor oil, POE-hydrogenated castor oil isostearate, and like POE-hydrogenated castor oil fatty acid esters; POE-octylphenyl ether and like POE-alkylaryl ethers; monoisostearyl glyceryl ether, monomyristyl glyceryl ether, and like glycerol alkyl ethers; POE-monostearyl glyceryl ether, POE-monomyristyl glyceryl ether, and like POE-glycerol alkyl ethers; diglycerol monostearate, decaglycerol decastearate, decaglycerol decaisostearate, diglycerol diisostearate, and polyglycerol fatty acid esters; sorbitan sesquioleate; and various other nonionic surfactants; lecithin, hydrogenated lecithin, saponin, sodium surfactin, cholesterol, bile acid, and like natural surfactants; and amino acid-based surfactants; etc. Such surfactants may be used singly. or in combination.

When using surfactant(s), the proportion thereof is not limited as long as the surfactant(s) have no adverse effects on the skin or mucosa and do not impair the effects of the present invention. The proportion can be suitably selected within the range of, for example, 0.01 to 30 wt. % of the total amount of the composition. From the viewpoint of the stability of active ingredients in the composition of the present invention and the feel on the skin, the proportion of surfactant(s) is preferably 0.1 to 20 wt. %, and more preferably 0.1 to 10 wt. %.

Usable gelling agents include, for example, carboxyvinyl polymers, polyoxyethylene-polyoxypropylene block copolymers, polyvinyl alcohol, sodium polyacrylate, sodium arginine, acrylate/alkyl methacrylate copolymers, etc. Such gelling agents can be used singly or in combination.

When using gelling agent(s), the proportion thereof is not limited as long as the gelling agent(s) have no adverse effects on the skin or mucosa and do not impair the effects of the present invention. The proportion can be suitably selected within the range of, for example, 0.01 to 20 wt. % of the total amount of the composition. From the viewpoint of the stability of active ingredients in the composition of the present invention and the feel on the skin, the proportion of gelling agent(s) is preferably 0.1 to 10 wt. %, and more preferably 0.5 to 5 wt. %.

Oils and/or fats that are used as components of compositions for external use in the field of pharmaceuticals, quasi drugs, or cosmetics, can be used without limitation. Examples of usable oils and fats include medium-chain fatty acid triglycerides and like synthetic oils and fats; soybean oil, rice oil, rapeseed oil, cottonseed oil, sesame oil, safflower oil, castor oil, olive oil, macadamia nut oil, cacao oil, camellia oil, sunflower oil, palm oil, linseed oil, perilla oil, shea oil, palm oil, japan wax, jojoba oil, grape seed oil, avocado oil, and like plant oils and fats; egg yolk oil, beef tallow, milk fat, lard, and like animal oils and fats; bees wax, spermaceti, lanolin, carnauba wax, candelilla wax, and like waxes; liquid paraffin, squalene, squalane,. microcrystalline wax, ceresin wax, paraffin wax, petrolatum, and like hydrocarbons; lauric acid, myristic acid, stearic acid, oleic acid, isostearic acid, behenic acid, and like natural and synthetic fatty acids; cetanol, stearyl alcohol, hexyldecanol, octyldecanol, lauryl alcohol, behenyl alcohol, and like natural and synthetic higher alcohols; isopropyl myristate, isopropyl palmitate, octyldodecyl myristate, octyldodecyl oleate, glyceryl tri-2-ethylhexanoate, octyl isononanoate, cholesterol oleate, and like esters and ethers; methylphenyl polysiloxane and like silicone oils; etc. Such oils and fats may be used singly or in combination.

When using oil(s) and/or fat(s), the proportion thereof is not limited as long as the oil(s) and/or fat(s) have no adverse effects on the skin or mucosa and do not impair the effects of the present invention. The proportion can be suitably selected within the range of, for example, 0.01 to 70 wt. % of the total amount of the composition. From the viewpoint of the stability of active ingredients in the composition of the present invention and the feel on the skin, the proportion of oil(s) and/or fat(s) is preferably 0.1 to 60 wt. %, and more preferably 0.1 to 50 wt. %.

Chelating agents that are used as components of compositions for external use in the field of pharmaceuticals, quasi drugs, or cosmetics, can be used without limitation. Usable chelating agents include, for example, ethylenediaminetetraacetic acid, ascorbic acid, citric acid, phytic acid, polyphosphoric acid, metaphosphoric acid, succinic acid, and salts thereof. Among such chelating agents, preferable examples are ethylenediaminetetraacetic acid, citric acid, and salts thereof, with ethylenediaminetetraacetic acid and salts thereof being particularly preferable.

Usable salts of ethylenediaminetetraacetic acid are not limited as long as they are pharmaceutically, pharmacologically, or physiologically acceptable, and include, for example, alkali metal salts such as sodium ethylenediaminetetraacetate, sodium ethylenediaminetetraacetate (trisodium edetate), disodium ethylenediaminetetraacetate, tetrasodium ethylenediaminetetraacetate, etc. Ethylenediaminetetraacetic acid and salts thereof can also be used in the form of hydrates. Examples of such hydrates include disodium ethylenediaminetetraacetate dihydrate (hereinafter sometimes-referred to as sodium edetate).

Such chelating agents can be used singly or in combination.

When using chelating agent(s), the proportion thereof is not limited as long as the chelating agent(s) have no adverse effects on the skin or mucosa and do not impair the effects of the present invention. The proportion can be suitably selected within the range of, for example, 0.0005 to 0.5 wt. % of the total amount of the composition. From the viewpoint of the stability of active ingredients in the composition of the present invention and the feel on the skin, the proportion of chelating agent(s) is preferably 0.001 to 0.2 wt. %, and more preferably 0.01 to 0.1 wt. %.

Saccharides that are used as components of preparations for external use in the field of pharmaceuticals, quasi drugs, or cosmetics, can be used without limitation. Examples of usable saccharides include monosaccharides (e.g., glucose, galactose, mannose, ribose, arabinose, xylose, deoxyribose, fructose, ribulose, lyxose, etc.), disaccharides (e.g., sucrose, trehalose, lactose, maltose, cellobiose, etc.), oligosaccharides (e.g., lactulose, raffinose, pullulan, etc.), cellulose or derivatives thereof (e.g, methylcellulose, ethylcellulose, hydroxyethylcellulose, hydroxypropylcellulose, hydroxypropylmethylcellulose, carboxymethylcellulose, carboxyethylcellulose, nitrocellulose, cationized cellulose, etc.); other polysaccharides [e.g, chondroitin sulfuric acid, dermatan, heparan, heparin, keratan, and salts thereof (e.g., sodium chondroitin sulfate, dermatan sulfate, heparan sulfate, keratan sulfate, and like pharmaceutically or physiologically acceptable salts), etc.], sugar alcohols (e.g., mannitol, xylitol, erythritol, pentaerythritol, maltitol, sorbitol, polydextrose, etc.), xylose, inositol, dextrin or derivatives thereof (dextrin palmitate and the like), honey, black sugar extract, etc. Such saccharides may be used singly or in combination.

UV protection agents that are used as components of compositions for external use in the field of pharmaceuticals, quasi drugs, or cosmetics, can be used without limitation. For example, zinc oxide, titanium oxide (crystal form: anatase, rutile, or brookite), kaoline, calcium carbonate, iron oxide, cerium oxide, zirconium oxide, titanium silicate, zinc silicate, silicic anhydride, cerium silicate, and like inorganic compounds; inorganic powders, such as mica, talc, etc., that are coated with the above-mentioned inorganic compounds; composites of the above-mentioned inorganic compounds and powders of resins such as polyamides, polyethylenes, polyesters, polystyrenes, nylons, etc.; the above-mentioned inorganic powders and composites that are further treated with silicone oil, aluminum salt of fatty acid, etc.; methyl diisopropylcinnamate, cinoxate, glyceryl diparamethoxycinnamate-mono-2-ethylhexanoate, mixture of isopropyl paramethoxycinnamate and diisopropylcinnamate, 2-ethylhexyl paramethoxycinnamate, benzyl cinnamate, and like cinnamic acid-type UV absorbers; oxybenzone, hydroxymethoxybenzophenone sulfonic acid, sodium hydroxymethoxybenzophenone sulfonate, dihydroxydimethoxybenzophenone, sodium dihydroxydimethoxybenzophenone disulfonate, dihydroxybenzophenone, tetrahydroxybenzophenone, and like benzophenone-type UV absorbers; paraaminobenzoic acid, ethyl paraaminobenzoate, glyceryl paraaminobenzoate, amyl paradimethylaminobenzoate, 2-ethylhexyl paradimethylaminobenzoate, ethyl 4-[N,N-di(2-hydroxypropyl)amino]benzoate, and like benzoate-type UV absorbers; ethylene glycol salicylate, octyl salicylate, dipropylene glycol salicylate, phenyl salicylate, homomenthyl salicylate, methyl salicylate, and like salicylic acid-type UV absorbers; guaiazulene, 2-ethylhexyl dimethoxybenzylidene dioxoimidazolidine propionate, 2,4,6-tris[4-(2-ethylhexyloxycarbonyl)anilino]1,3,5-triazine, parahydroxyanisole, 2-(2-hydroxy-5-methylphenyl)benzotriazol, 4-tert-butyl-4′-methoxydibenzoylmethane, etc. Such UV protection agents may be used singly or in combination.

The composition of the present invention may further contain, as required, various components that are generally used as components of compositions for external use in the field of pharmaceuticals, quasi drugs, or cosmetics, such as irritation reducing agents, thickeners, preservatives, coloring agents, dispersants, pH adjusters, perfumes, etc. Such components are used within quantitative and qualitative ranges that do not deteriorate the properties, such as appearance stability, viscosity, etc., and that do not impair the effects of the present invention. Such components can be used singly or in combination.

The composition of the present invention usually has a pH of 1 to 8, but from the viewpoint of the stability of the composition, low irritation to the skin and mucosa, and pleasant feel on the skin, it is desirable that the composition have an acidic pH, i.e., preferably 2 to 7, and more preferably 2 to 6.

The form of the composition of the present invention is not limited as long as the composition can be used on the skin, and may be, for example, a paste, mousse, gel, liquid, emulsion, cream, or the like. The composition of the present invention may be supported on a predetermined substrate or accommodated in a predetermined container so that it can be used as a sheet-type preparation, aerosol preparation, or spray preparation.

The composition of the present invention can exhibit various cosmetic and pharmacological actions based on the physiological activity of hydroquinone, and thus can be used as a cosmetic composition, skin cleansing composition, or pharmaceutical composition.

Examples of cosmetic compositions include milky lotions, creams, lotions, oils, packs, and like cosmetic compositions for basic skin care; UV protection cosmetic compositions; skin-whitening cosmetic compositions; etc.

Examples of skin cleansing compositions include face cleansing preparations, makeup removers, body cleansing preparations, etc.

Examples of pharmaceutical compositions include those for treating chloasmas, acne scars, and freckles.

The composition of the present invention effectively exhibits the melanogenesis inhibitory effect, i.e., skin-whitening effect, of hydroquinone, while reducing cytotoxicity and alleviating skin-irritating properties. The composition is therefore safe to apply on the skin everyday. In view of such effects of the present invention, the composition of the present invention is particularly useful as a cosmetic composition, and more particularly as a skin-whitening cosmetic composition, among the above-mentioned uses.

The composition of the present invention is used by applying on the skin. The method for applying the composition of the present invention on the skin can be suitably selected according to the form, intended use, etc., of the composition. For example, a suitable amount of the composition of the present invention is applied on the skin by'spreading, spraying, or using patches, once to several times a day.

The composition of the present invention can be prepared by mixing, with a pharmaceutically or cosmetically acceptable carrier, predetermined amounts of hydroquinone, tocopherol(s), and if necessary, additional components as mentioned above.

### 2. Method for Reducing Cytotoxicity of Hydroquinone-Containing Composition for External Use

The present invention further provides a method for reducing cytotoxicity of a hydroquinone-containing composition for external use. Specifically, the method is carried out by adding tocopherol(s) to a hydroquinone-containing composition for external use.

The hydroquinone and tocopherols used in the method; the proportions thereof in the composition for external use; other components that can be used in the composition; and the form, intended use, etc., of the composition are the same as those described in “1. Composition for External Use” given above.

### 3. Method for Reducing Cytotoxicity of Hydroquinone on the Skin

In addition, the present invention provides a method for reducing cytotoxicity of hydroquinone on the skin. Specifically, the method is carried out by applying a composition (A) containing hydroquinone and a composition (B) containing at least one tocopherol to the skin.

There is no limitation on the order of applying the compositions (A) and (B). The method may be carried out by applying the composition (A) together with composition (B) to the skin, by applying the composition (B) to the skin after applying the composition (A), or applying the composition (A) to the skin after applying the composition (B). Preferable is a method comprising applying the composition (A) to the skin after applying the composition (B).

The hydroquinone and tocopherols used in the method are the same as those described in “1. Composition for External Use” given above.

The proportion of hydroquinone in the composition (A); and the form, intended use, etc., of the composition (A) are the same as those described in “1. Composition for External Use” given above.

The proportion of tocopherol in the composition (B); and the form, intended use, etc., of the composition (B) are the same as those described in “1. Composition for External Use” given above.

Further, the ratio of the tocopherol to hydroquinone on the skin, when applied compositions (A) and (B), are the same as those described in “1. Composition for External Use” given above.

### EXAMPLES

The following Test Examples and Examples illustrate the present invention in further detail, and are not intended to limit the present invention. In the formulations shown below, percentages are by weight (W/W) unless otherwise indicated.

### Test Example 1

### Cytotoxicity Evaluation Test

Normal human dermal fibroblasts (NHDF-AD: product of Kurabo Industries, Ltd.) were inoculated onto a 24-well plate (1.0×10⁴ cells/well), and incubated for four days in 1 ml of DMEM containing 10% FBS(vol/vol). Subsequently, the supernatant of the wells was replaced with 1 ml of fresh DMEM containing 10% FBS(vol/vol), and 5 μl of ethanol solution containing a predetermined concentration of tocopherol or 5 μl of ethanol was added to the concentrations shown in Tables 1 and 2, and 20 ml of 0.045% hydroquinone solution was added, followed by incubation for 24 hours. Then, the supernatant of the wells were removed by replacement with 1 ml of medium, and the number of cells that survived was determined by WST-1 assay. From the results, the cell survival rates were calculated, with the number of cells that survived in Control 1 being set at 100.

Tables 1 and 2 show the results.

TABLE 1

| g/100 ml | Example 1 | Example 2 | Example 3 | Example 4 | Example 5 | Control 1 |
|---|---|---|---|---|---|---|
| Hydroquinone | 0.0009 | 0.0009 | 0.0009 | 0.0009 | 0.0009 | 0.0009 |
| α-Tocopherol | 0.003 | 0.01 | — | — | — | — |
| γ-Tocopherol | — | — | 0.0003 | 0.001 | 0.003 | — |
| Cell survival | 125 | 175 | 225 | 250 | 300 | 100 |
| rate | | | | | | |

TABLE 2

| g/100 ml | Example 6 | Example 7 | Example 8 | Example 9 | Control 1 |
|---|---|---|---|---|---|
| Hydro- | 0.0009 | 0.0009 | 0.0009 | 0.0009 | 0.0009 |
| quinone | | | | | |
| δ- | 0.0003 | 0.001 | 0.003 | 0.01 | — |
| Tocopherol | | | | | |
| Cell survival | 338 | 413 | 450 | 488 | 100 |
| rate | | | | | |

Table 1 reveals that the cells to which hydroquinone and α-tocopherol had been added had a survival rate more than 1.3 times higher than that of Control 1. Table 1 also shows that the cells to which hydroquinone and γ-tocopherol had been added had a survival rate more than 2.3 times higher than that of Control 1. It is confirmed from these results that α-tocopherol and γ-Tocopherol have hydroquinone-cytotoxicity-reducing effect.

Table 2 shows that the cells to which hydroquinone and δ-tocopherol had been added had a remarkably higher survival rate than that of Control 1, even when the concentration of δ-tocopherol was low. In particular, in Example 6, although the concentration of δ-tocopherol was as low as 0.0003 g/100 ml, the cell survival rate was about 3.4 times higher than that of Control 1.

Thus, all Examples demonstrated the effect of reducing hydroquinone cytotoxicity.

### Test Example 2

### Cytotoxicity Evaluation Test

Using normal human epidermal keratinocytes (NHEK-F, product of Kurabo Industries, Ltd.), the procedure of Test Example 1 was followed to evaluate the cytotoxicity except that the medium used was EpiLife-HKGS (product of Kurabo Industries, Ltd.), and the number of cells inoculated was 2.0×10⁴ cells/well. From the results, the cell survival rates were calculated, with the number of cells that survived in Control 2 being set at 100.

Table 3 shows the results.

TABLE 3

| | Example | Example | Example | |
|---|---|---|---|---|
| g/100 ml | 10 | 11 | 12 | Control 2 |
| Hydroquinone | 0.001 | 0.001 | 0.001 | 0.001 |
| γ-Tocopherol | 0.0001 | — | — | — |
| δ-Tocopherol | — | 0.00005 | 0.0001 | — |
| Cell | 130 | 123 | 137 | 100 |
| survival | | | | |
| rate | | | | |

In all Examples, the rate of cell survival after addition of hydroquinone was higher than that of Control 2. Further, the p values in Examples 10, 11, and 12 were 0.001%, 0.02%, and 0.001%, respectively, which are statistically significant.

Thus, all Examples demonstrated the effect of reducing hydroquinone cytotoxicity.

Although α-tocopherol is known to have the highest physiological activity among known tocopherols, Test Examples 1 and 2 showed unexpected results that, in both fibroblasts and epidermal keratinocytes, which are present in the skin, δ-tocopherol exhibits the highest hydroquinone-cytotoxicity-reducing activity, and γ-tocopherol exhibits the second highest hydroquinone-cytotoxicity-reducing activity. In particular, the test using epidermal keratinocytes indicates the high safety of the composition of the present invention on the skin, since the epidermal keratinocytes used in Test Example 2 were tested at a higher concentration than fibroblasts used in Test Example 1, and the excellent effects were demonstrated using epidermal keratinocytes which are main cellular components of the epidermis. In view of Test Examples 1 and 2, it is clear that the composition of the present invention has high safety on the skin.

Further, in the composition for external use of the present invention, primary skin irritation caused by hydroquinone are also reduced.

### Examples 13-22

Examples of formulations are given below. The percentages in the following Examples were by weight unless otherwise indicated.

### Example 13

### Beauty Lotion

| | (wt. %) |
|---|---|
| Hydroquinone | 4 |
| δ-Tocopherol | 2 |
| Sodium ascorbate | 0.5 |
| Sage extract | 0.1 |
| 1,3-Butylene glycol | 15 |
| Diglycerol | 55 |
| Purified water | q.s. 100 |
| | 100 |

### Example 14

### Beauty Lotion

| | (wt. %) |
|---|---|
| Hydroquinone | 1 |
| γ-Tocopherol | 2 |
| Magnesium ascorbyl phosphate | 3 |
| Dipropylene glycol | 40 |
| Propylene glycol | 20 |
| Purified water | q.s. 100 |
| | 100 |

### Example 15

### Lotion

| | (wt. %) |
|---|---|
| Hydroquinone | 2 |
| γ-Tocopherol | 1 |
| Pyridoxine hydrochloride | 0.1 |
| Dipotassium glycyrrhizinate | 0.2 |
| Glycerol | 15 |
| Ethanol | 10 |
| 1,3-Butylene glycol | 5 |
| Polyoxyethylene hydrogenated castor oil | 1 |
| Lactic acid | 1 |
| Sodium arginate | 0.1 |
| Purified water | q.s. 100 |
| | 100 |

### Example 16

### Pack

| | (wt. %) |
|---|---|
| Hydroquinone | 1 |
| γ-Tocopherol | 2 |
| Mulberry bark extract | 0.1 |
| Diglycerol | 20 |
| 1,3-Butylene glycol | 10 |
| Polyvinyl alcohol | 10 |
| Ethanol | 8 |
| Ethylene glycol | 0.4 |
| Methylparaben | 0.1 |
| Perfume | 0.2 |
| Purified water | q.s. 100 |
| | 100 |

### Example 17

### Milky Lotion

| | (wt. %) |
|---|---|
| Hydroquinone | 8 |
| γ-Tocopherol | 6 |
| Squalane | 2 |
| Aloe extract | 0.5 |
| Glycerol | 15 |
| Diglycerol | 10 |
| Polyoxyethylene oleyl ether | 2.8 |
| Sesquioleic acid sorbitan | 2.2 |
| Octyl isononanoate | 1 |
| Carboxyvinyl polymer | 0.1 |
| Potassium hydroxide | 0.1 |
| Trisodium edetate | 0.1 |
| Phenoxyethanol | 0.1 |
| Methylparaben | 0.15 |
| Butylparaben | 0.05 |
| Purified water | q.s. 100 |
| | 100 |

### Example 18

### Cream

| | (wt. %) |
|---|---|
| Hydroquinone | 4 |
| δ-Tocopherol | 4 |
| Arbutin | 1 |
| Parsley extract | 0.5 |
| Pyridoxine hydrochloride | 0.1 |
| Retinol | 0.01 |
| White petrolatum | 20 |
| Diglycerol | 15 |
| Stearyl alcohol | 10 |
| Propylene glycol | 5 |
| Behenyl alcohol | 5 |
| Polyoxyethylene(60) | 4 |
| hydrogenated castor oil | |
| Glycerol monostearate | 1 |
| Methylparaben | 0.1 |
| Propylparaben | 0.1 |
| Dibutylhydroxytoluene | 0.01 |
| Purified water | q.s. 100 |
| | 100 |

### Example 19

### Beauty Lotion

| | (wt. %) |
|---|---|
| Hydroquinone | 1 |
| Arbutin | 1 |
| γ-Tocopherol | 2 |
| Sodium ascorbate | 3 |
| Dipropylene glycol | 40 |
| Propylene glycol | 20 |
| Purified water | q.s. 100 |
| | 100 |

### Example 20

### Sunscreen Cream

| | (wt. %) |
|---|---|
| Hydroquinone | 1 |
| γ-Tocopherol | 4 |
| Hydroxyproline | 0.5 |
| Zinc oxide | 10 |
| Glyceryl tri-2-ethylhexanoate | 10 |
| Liquid paraffin | 8 |
| Dipropylene glycol | 5 |
| Octyl paramethoxycinnamate | 5 |
| Olive oil | 5 |
| Oxybenzone | 4 |
| Triethanolamine | 0.5 |
| Acrylate/ | 0.4 |
| alkyl methacrylate copolymer | |
| Carboxyvinyl polymer | 0.2 |
| Phenoxyethanol | 0.2 |
| Purified water | q.s. 100 |
| | 100 |

### Example 21

### Gel

| | (wt. %) |
|---|---|
| Hydroquinone | 0.5 |
| δ-Tocopherol | 1.0 |
| Hamamelis extract | 1.5 |
| Allantoin | 0.1 |
| Propylene glycol | 15 |
| 1,3-Butylene glycol | 5 |
| Polyoxyethylene (60) | 2 |
| hydrogenated castor oil | |
| Carboxyvinyl polymer | 0.5 |
| Potassium hydroxide | 0.35 |
| Hydroxypropylmethylcellulose | 0.2 |
| Methylparaben | 0.2 |
| Hydrogenated soybean phospholipid | 0.05 |
| Sodium edetate | 0.02 |
| Sodium polyacrylate | 0.01 |
| Purified water | q.s. 100 |
| | 100 |

### Example 22

### Beauty Lotion

| | (wt. %) |
|---|---|
| Hydroquinone | 2 |
| α-Tocopherol | 2 |
| Sodium ascorbate | 0.5 |
| Diglycerol | 55 |
| 1,3-Butylene glycol | 15 |
| Purified water | q.s. 100 |
| | 100 |

1. A composition for external use comprising hydroquinone and at least one tocopherol.

2. A composition according to claim 1, wherein said least one tocopherol is selected from the group consisting of α-tocopherol, γ-tocopherol, and δ-tocopherol.

3. A composition according to claim 1, wherein said at least one tocopherol is selected from the group consisting of γ-tocopherol and δ-tocopherol.

4. A composition according to claim 1, comprising 0.0005 to 10 wt. % of hydroquinone and 0.01 to 10 wt. % of tocopherol, based on the total amount of the composition.

5. A composition according to claim 1, comprising 0.01 to 12 parts by weight of tocopherol per part by weight of hydroquinone.

6. A composition according to claim 1, which is a cosmetic composition.

7. A composition according to claim 1, which is a skin cleansing composition.

8. A composition according to claim 1, which is a pharmaceutical composition.

9. A process for producing a composition for external use, the process comprising the step of adding hydroquinone and at least one tocopherol to a pharmaceutically or cosmetically acceptable carrier.

10. A process according to claim 9, wherein said at least one tocopherol is selected from the group consisting of α-tocopherol, γ-tocopherol, and δ-tocopherol.

11. A process according to claim 9, wherein said least one tocopherol is selected from the group consisting of γ-tocopherol and δ-tocopherol.

12. A process according to claim 9, wherein the hydroquinone and tocopherol are added in proportions of 0.0005 to 10 wt. % and 0.01 to 10 wt. %, respectively, based on the total amount of the composition.

13. A process according to claim 9, wherein the hydroquinone and tocopherol are added in a ratio of 0.01 to 12 parts by weight of tocopherol per part by weight of hydroquinone.
